(19) United States
(12) Patent Application Publication
(10) Pub. No.: US 2001/0006646 A1
(43) Pub. Date: Jul. 5, 2001

(54) METHOD AND SYSTEM FOR TREATMENT OF WOUNDS

(76) Inventor: KATHLEEN A COYNE, PITTSBURGH, PA (US)

Correspondence Address: ECKERT SEAMANS CHERIN & MELLOTT, LLC, SUITE 3232, 1515 MARKET STREET, 9TH FLOOR, PHILADELPHIA, PA, 19102

(21) Appl. No.: 09/012,618

(22) Filed: Jan. 23, 1998

Related U.S. Application Data
(63) Continuation-in-part of application No. 08/775,018, filed on Dec. 27, 1996, now abandoned.

(51) Int. Cl. A61K 9/00

(52) U.S. Cl. 424/400

(57) ABSTRACT

Formulations and methods using such formulations for stimulating wound healing of skin and related tissues, particularly healing of pressure wounds and other chronic or intractable wounding conditions. More particularly, the present invention provides formulations which stimulate the healing of wounds and which consist essentially of a pharmaceutically effective combination of insulin, hydrocortisone and a nutrient filled medium and methods of using the same.

### BACKGROUND OF THE INVENTION

1. Field of the Invention

The present invention relates to formulations and methods using such formulations for stimulating wound healing of skin and related tissues, particularly healing of pressure wounds and other chronic or intractable wounding conditions. More particularly, the present invention relates to formulations which stimulate the healing of wounds and which consist essentially of a pharmaceutically effective combination of insulin, hydrocortisone and a nutrient filled medium and methods of using the same.

2. Background of the Invention

The skin of patients, including humans, non-human mammals and reptiles, provides a variety of critical homeostatic functions and, by virtue of its location on the body's surface, is exposed to a variety of insults which may result in wounds. Such wounds may include, for example, cuts, abrasions, thermal and chemical burns, chemical exposure, radiation burns, surgical incisions, disease conditions, and the like. Furthermore, there are a number of chronic or intractable wounding conditions which result in delayed healing, for example, chronic bed sores. There are also certain disease states, for example, diabetes, which are accompanied by a retardation in wound healing. Delayed healing results in an increased susceptibility to infection, a prolonged recovery period, an increase in the cost of treatment, and an increase in mortality.

Bed sores (known as decubitus ulcers by the medical profession) are characterized by a chronic, morbid condition of the skin which often afflicts the disabled, the elderly and the bedridden. Often, bedsores are under treatment for months and sometimes even years in an attempt to effectuate healing. Traditional treatments for bedsores, implemented with limited success, using a variety of means include the application of collagen, maalox & sugar, antacids, Betadine, topical & oral antibiotics, transparent films, freeze dried gels, pressure dressings, absorptive dressings, alginates, cleansers, contact layers, enzymatic debriders, fillers, foam dressings, growth factors, hydro colloid, moisturizers, support surface devices, etc.

Chronic or intractable wounding conditions present a concern both for the patient afflicted therewith and for the institution treating the patient. Specifically, such conditions result in tremendous expense to the state and federal government to cover the cost of caring for such wounds. For example, an estimated $23 billion dollars is spent annually to pay for conventional bed sore treatments for human patients in the United States alone. Accordingly, it would be desirable, for both the practice of veterinary and human medicine, to provide methods and pharmaceutical formulations which stimulate wound healing in patients. Particularly, it would be desirable to provide methods and pharmaceutical formulations which stimulate the healing in patients of otherwise non-healing wounds.

### SUMMARY OF THE INVENTION

Briefly, the compositions and procedures of the present invention are useful in stimulating and accelerating the healing of wounds to the skin of human, non-human mammal and reptilian patients. In particular the present invention has been observed to be useful for stimulating and accelerating the natural wound healing process in human, non-human mammal and reptilian patients. The formulations and methods of the present invention have also been observed to stimulate the healing of chronic or intractable wounding conditions, e.g., pressure wounds. The formulations of the present invention generally comprise a combination of insulin, hydrocortisone and a nutrient filled medium. The methods of the present invention generally comprise topically administering to a wound a therapeutically effective amount of a formulation comprised of insulin, hydrocortisone and a nutrient filled medium.

In preferred embodiments according to the present invention, the formulations include insulin, preferably human insulin of a purity suitable for intravenous injection in a human patient in combination with hydrocortisone, preferably hydrocortisone succinate suitable for intravenous infusion or intramuscular injection in a human patient. In general, the insulin and hydrocortisone are included in therapeutically effective amounts in the formulations of the present invention. The preferred amounts of insulin and hydrocortisone to be used is generally dependent upon the type and size of the wound. In formulations for treating pressure wounds, e.g., decubitus ulcers, insulin will preferably be present in an amount equivalent to at least about 200 Units of insulin per cubic centimeter of wound in combination with hydrocortisone preferably present in an amount equivalent to at least about 500 mg per cubic centimeter-meter of wound.

In an embodiment of the present invention, the nutrient filled medium includes vitamins, proteins, lipids, carbohydrates and oils. Preferably, the nutrient filled medium includes all of the nutrients needed by the cells within the wound to fuel the healing process.

In an embodiment of the present invention, the formulations may optionally include topical anesthetics. Preferably, the formulations of the present invention contain pramoxine hydrochloride in over the counter strength.

In an embodiment of the present invention, the formulations may optionally include preservatives. Preferably, the formulations of the present invention contain at least one of Glydant Plus®, methyl paraban, silicone 5-5-6, propylparaban, BHT, and C₁₂₋₁₅ alkyl benzoate.

In an embodiment, the present invention provides a medicated pad consisting of a sterile pad, preferably a sterile gel pad with a Telfa-type covering, impregnated with a formulation of the present invention. Particularly, in this embodiment, a medicated pad is provided consisting of a sterile pad impregnated with a therapeutically effective amount of a formulation of the present invention. Optionally, the medicated pad may further comprise a self adhesive bandage. Specifically, the medicated pad may optionally have means for securing itself to a patient. For example, the medicated pad may optionally have an adhesive strip attached thereto at or near its periphery which extends along at least a portion of the periphery and, optionally, may project outwardly therefrom. Also, the medicated pads of the present invention may be provided in a ready-to-use state. For example, the medicated pads may be supplied and stored in hermetically sealed containers until needed, at which time the medicated pads may be removed from the sealed container and placed over a wound.

In an embodiment of the present invention, medicated pads are provided containing a suitable concentration of a formulation comprised of insulin, hydrocortisone, pramoxine hydrochloride, phenyl-epherine and a nutrient filled medium. The preferred amounts of insulin, hydrocortisone and other constituents of the formulations contained in the medicated pads of the present invention is generally dependent upon the type and size of the wound. For example, medicated pads intended for use in stimulating the healing of pressure wounds, e.g., bed sores, preferably have a concentration of insulin equivalent to at least 200 Units per cubic centimeter of wound to be treated and a concentration of hydrocortisone sodium succinate equivalent to at least 500 mg per cubic centimeter of wound to be treated. Medicated pads having a concentration of insulin equivalent to from about 150 units per cubic centimeter of wound to be treated up to about 1,000 units per cubic centimeter of wound to be treated are anticipated as effective.

The present invention also provides a method for stimulating the healing of wounds to the skin of human, non-human mammal and reptilian patients. In one embodiment, the present invention provides a method for stimulating wound healing by contacting the wound with a formulation comprising a therapeutically effective combination of insulin, hydrocortisone and a nutrient filled medium. Preferably, the formulation will be a topical pharmaceutical formulation consisting of a lipid solution, a gel, a foam, a cream, a paste, a lotion, a spray, a suspension, a dispersion, a salve or an ointment, most preferably a gel which may be readily absorbed into the patient's skin. In accordance with another aspect of this embodiment, the insulin is preferably human insulin of a purity suitable for intravenous injection into a human patient. In yet another aspect of this embodiment, the hydrocortisone is of a purity suitable for intravenous or intramuscular use by a human patient.

In an embodiment, the present invention provides a method for stimulating healing of a wound to the skin of a human, non-human mammal or reptilian patient, comprising: (a) preparing the wound for treatment; (b) removing from a hermetically sealed container a medicated pad impregnated with a formulation consisting of a pharmaceutically effective combination of insulin, hydrocortisone and a nutrient filled medium, wherein the preferred amounts of insulin, hydrocortisone and other constituents of the formulation are generally dependent upon the type and size of the wound to be treated; (c) covering the wound with the medicated pad; and (d) repeating steps (a) through (c) at least daily until the wound is healed.

### DETAILED DESCRIPTION OF THE PRESENTLY PREFERRED EMBODIMENTS

The term “wound” is used throughout the present specification to describe wounds to the skin of human, non-human mammal and reptilian patients which may be treated using the formulations and methods of the present invention. A wound to the skin constitutes a breach in the continuity of the skin tissue caused by some insult thereto. Wounds to the skin may include, without limitation, cuts, punctures, surgical incisions, lacerations, abrasions, chemical and thermal burns, radiation burns, disease conditions, and the like. The formulations and methods of the present invention are believed to be useful for stimulating and/or accelerating the healing of wounds to the skin of human, non-human mammal and reptilian patients, particularly in the case of chronic or intractable wounding conditions including pressure wounds, e.g., chronic bed sores, ulcerative skin conditions and other difficult to heal wounds. The formulations and methods of the present invention are also believed useful in treating the wounds of patients with disease states, for example diabetes, which are associated with retarded wound healing.

The phrase “therapeutically effective amount” is used herein in the qualitative sense to mean an amount sufficient to stimulate, to a clinically significant degree, the healing of wounds to the skin of a human, non-human mammal or reptilian patients. Quantitatively, with respect to pressure wounds, e.g., decubitus ulcers, and other chronic or intractable wounding conditions, the phrase means an amount sufficient to stimulate the healing of the wounds so as to effect closure of the wounds within approximately two to three weeks with daily treatments, preferably within about one week.

The phrase “amount effective to support wound healing” is used herein in the qualitative sense an amount sufficient to support the healing process for wounds to the skin of human, non-human mammal or reptilian patients. Quantitatively, with respect to pressure wounds, e.g., decubitus ulcers, and other chronic or intractable wounding conditions, the phrase means an amount sufficient to transport and supply nutrients required by the normal, distressed and injured cells which surround or comprise the wound, enhancing the growth and repair mechanisms believed to be responsible for the healing process effecting closure of the wounds within approximately two weeks with daily treatments, preferably within about one week.

The term “Unit” used herein to describe quantities of insulin is known in the art as the equivalent of an International Unit of Insulin. An International Unit of Insulin is defined as the activity contained in 0.04167 mg of the 4th International Standard Preparation (1958). The Standard Preparation being a quantity of purified Zinc Insulin crystals extracted 52% from Bovine and 48% from Porcine pancreas. Martindale Pharmacopeia, 26th Edition.

The formulations and methods of the present invention are specifically designed to stimulate the healing of wounds. Particularly, the formulations of the present invention are believed to stimulate the healing of wounds in patients, thereby reducing the risk of infection associated with delayed wound healing.

The formulations of the present invention for stimulating healing of wounds in human, non-human mammal and reptilian patients consist essentially of insulin, hydrocortisone and a nutrient filled medium.

The formulations of the present invention contain a pharmaceutically effective amount of insulin. Preferably, the insulin used in the formulations of the present invention is human insulin, most preferably human insulin of a purity suitable for intravenous use in a human patient. Human insulin is commercially available from a number of sources. For example, human insulin is distributed in the United States by Novo Nordisk Pharmaceuticals, Inc. under the trade name Novolin® and Eli Lilly under the trade name Iletin®.

The formulations of the present invention contain a pharmaceutically effective amount of hydrocortisone or hydrocortisone esters. The hydrocortisone is preferably in the form of a hydrocortisone ester, more preferably hydrocortisone sodium succinate, most preferably, hydrocortisone sodium succinate of a purity suitable for intravenous infusion or intramuscular injection in a human patient. Hydrocortisone sodium succinate suitable for use with the present invention is commercially available from UpJohn under the trade name Solucortef®.

The formulations of the present invention also include a nutrient filled medium which may contain vitamins, proteins, lipids, carbohydrates, salicylic acid complex, acetates, lipid protein complexes and oils.

Preferably, the vitamins included in the formulations of the present invention include a source of Vitamin A, a source of Vitamin C and a source of Vitamin E.

Vitamin A is included in the formulations of the present invention in an amount effective to support wound healing. Preferably, the formulations of the present invention include at least 400 IU of Vitamin A. Most preferably, the formulations of the present invention include an amount of Vitamin A equivalent to at least 100% of the recommended daily dietary allowance of Vitamin A for the patient. Preferably, the formulations of the invention include retinyl palminate as a source of Vitamin A.

Vitamin C is included in the formulations of the present invention in an amount effective to support wound healing. Preferably, the formulations of the present invention include at least 800 IU of Vitamin C. Most preferably, the formulations of the present invention will include an amount of Vitamin C equivalent to at least 100% of the recommended daily dietary allowance of Vitamin C for the patient. Preferably, the formulations of the present invention will include ascorbyl glucosamine as a source of Vitamin C.

Vitamin E is included in the formulations of the present invention in an amount effective to support wound healing. Preferably, the formulations of the present invention include an amount of Vitamin E equivalent to at least 100% of the recommended daily dietary allowance of Vitamin E for the patient. Preferably, the formulations of the present invention include at least 400 IU of tocopherol acetate and at least 400 IU of tocopherol linoleate. Most preferably, the formulations of the present invention include at least 400 IU of tocopherol acetate and at least 800 IU of tocopherol linoleate.

The formulations of the present invention preferably contain proteins in an amount effective to support wound healing. The proteins included in the formulations of the present invention may contain both essential and non-essential proteins. Preferably, the formulations of the invention comprise about 50 wt % proteins. Most preferably, the formulations of the present invention include a source of proteins equivalent to at least 15 % of the recommended daily dietary requirement for the patient. A variety of protein sources are suitable for use in the formulations of the present invention. For example, suitable protein sources include milk proteins; vegetable proteins; meat proteins; wheat glyceride; zinc wheat protein collagen extract from sea moss, oat collagen; purified wheat collagen; purified barley yeast; exanthum gum; carrageenan; babasu oil; cetyl-sterol-ethoxulate-ceteareth 15 moles, molecule; preferably wheat glyceride, oat collagen and zinc wheat protein collagen extract from sea moss.

The formulations of the present invention also preferably contain lipids in an amount effective to support wound healing. Preferably, the formulations of the present invention comprise at least 7 vol. % lipids. The lipids used in the formulations of the present invention preferably comprise, or may be derived from, long, medium, or short chain highly polyunsaturated oils, such as, babasu oil, avocado oil, macadamia oil, safflower oil, sunflower oil and cetyl ethanol, most preferably babasu oil.

The formulations of the present invention preferably also contain carbohydrates in an amount effective to support wound healing. The carbohydrates included in the formulations of the present invention preferably comprise both simple and complex carbohydrates. Preferably, the formulations of the present invention include a source of carbohydrates equivalent to at least 28% of the recommended daily dietary requirement for the patient. The carbohydrates used in the formulations of the present invention may comprise, or may be derived from, at least one of oat colloids, purified oat colloids maltodextrin, sucrose, corn starch, corn syrup solids, wheat glyceride, glycerol sterate, protein extracted from sea moss, barley extract, exanthum gum, glycerine and glucosamine.

The formulations of the present invention preferably also contain oil in an amount effective to support wound healing. The oil included in the formulations of the present invention preferably comprise hyper-polyunsaturated oils or mixtures thereof. Preferably, the formulations of the present invention comprise at least 7 vol % oil(s). Most preferably, the formulations of the invention comprise 7 to 10 vol % oil(s). The oil(s) used in the formulations of the present invention may comprise, or may be derived from, at least one of macadamia oil, avocado oil, emu oil, babasu oil, sunflower oil, and safflower oil.

The formulations of the present invention may also include at least one polymer vehicle selected from the group of collagen, gelatin, polyethylene glycol, polyvinylpyrrolidone, glycerolmethacrylate and hydroxyethylmethacrylate.

The formulations of the present invention may also include preservatives to help extend the shelf-life thereof. Preferably, the formulations of the present invention include at least one of Glydant Plus®, methyl paraban, propylbaraban, BHT, and C₁₂₋₁₅ alkyl benzoates. Most preferably, the formulations of the present invention include Glydant Plus®.

The formulations of the present invention may also include antioxidants to prevent rancidity of the oils present in the formulation. Preferably, the formulations of the invention include Tenox-6®. Other antioxidants believed to be suitable for use in the formulations of the invention include β-hydroxy anti-oxidants and α-hydroxy anti-oxidants. Those skilled in the art will know how to determine the quantity of antioxidant(s) to include in the formulations to protect against rancidity of the oils included in a given formulation.

The formulations of the present invention may also optionally include antibacterial agents, antifungal agents, antiosmotic agents and similar materials in conventional quantities used in topical applications. These materials may be added to the formulation for their ability to treat a given infection or as a prophylactic against infection.

Generally speaking, the amount of each component present in the formulations of the present invention will depend on the type and size of the wound to be treated. For instance, for pressure wounds, the formulations of this preferred aspect of the invention include per cubic centimeter of wound at least 200 Units of insulin and at least 500 mg of hydrocortisone.

The formulations of the present invention may be provided as topical pharmaceutical compositions consisting of a lipid solution, a gel, a foam, a cream, a paste, a lotion, a spray, a suspension, a dispersion, a salve or an ointment, most preferably a gel which may be readily absorbed into the patient's skin. Those skilled in the art will know how to prepare topical pharmaceutical compositions based on the formulations of the present invention.

In another embodiment, the present invention provides medicated pads consisting of a sterile pad impregnated with a formulation of the present invention. Most preferably, the sterile pad comprises a pad having a telfa-type back padding. Optionally, the medicated pad may further comprise means for securing it to the patient. Preferably, the means for securing comprises an adhesive strip attached to the medicated pad at or near its periphery which adhesive strip extends along at least a portion of the periphery and, optionally, may project outwardly therefrom. Also, the medicated pads of the present invention may preferably be provided in a ready-to-use fashion. For example, the medicated pads may be supplied and stored in hermetically sealed containers until needed, at which time the medicated pads may be removed from the sealed containers and placed over a wound.

In a preferred aspect of this embodiment, medicated pads may be provided which contain a pharmaceutically effective amount of a formulation of the invention suitable for use in stimulating healing of a given type, size and severity of wound, e.g., pressure wounds. In this aspect, the medicated pads are prepackaged and stored, prior to use, in ready-to-use fashion in hermetically sealed containers labeled for use on specific types, sizes and severities of wounds.

In another embodiment, the present invention provides a method for stimulating the healing of wounds to the skin of human, non-human mammal and reptilian patients. In one aspect of this embodiment, the present invention provides a method for stimulating wound healing by contacting the wound with a formulation comprising a therapeutically effective combination of insulin, hydrocortisone and a nutrient filled medium. Preferably, the formulation will be a topical pharmaceutical composition consisting of a lipid solution, a gel, a foam, a cream, a paste, a lotion, a spray, a suspension, a dispersion, a salve or an ointment, most preferably a gel which may be readily absorbed into the patient's skin. In accordance with another aspect of this embodiment, the insulin is preferably human insulin of a purity suitable for intravenous injection into a human patient. In yet another aspect of this embodiment, the hydrocortisone is of a purity suitable for intravenous or intramuscular use by a human patient. Those skilled in the art will know how to prepare topical pharmaceutical compositions based on the formulations of the present invention.

In another embodiment, the present invention provides a method for stimulating healing of a wound to the skin of a human, non-human mammal or reptilian patient, comprising: (a) preparing the wound for treatment; (b) removing from a hermetically sealed container a medicated pad impregnated with a formulation consisting of a pharmaceutically effective combination of insulin, hydrocortisone and a nutrient filled medium, wherein the preferred amounts of insulin, hydrocortisone and other constituents of the formulation are generally dependent upon the type and size of the wound to be treated; (c) covering the wound with the medicated pad; and (d) repeating steps (a) through (c) at least daily, until the wound is healed. For example, in the case of years old, chronically non-healing, pressure wounds, wound closure is typically achieved after about one to two weeks of daily treatments.

In this embodiment, the wound preparation step comprises (i) removing any dressing which may be covering the wound; (ii) application of a chemical debridement compound, preferably an aerosolized, premeasured dose of a chemical debriding complex, namely a liquid composition comprising:

sterile babasu oil,

sterile avocado oil,

(3%) hydrogen peroxide,

pramoxine hydrochloride (over the counter strength), and

optionally, povidone iodine; and,

(iii) waiting for the chemical debridement compound to bubble off, preferably at least three (3) minutes.

The medicated pads used to cover the wound in this embodiment are preferably impregnated with a pharmaceutically effective amount of a formulation of the present invention for use on the type of wound being treated and provided in ready-to-use fashion in hermetically sealed containers which are labeled for use on the specific type and severity of wound which is being treated. Also, the medicated pads are preferably provided with means for securing them to the patient. Thus, a health care practitioner may select an appropriately labeled container, remove the medicated pad therefrom, cover the wound with the medicated pad and secure the medicated pad to the patient.

Preliminary bioassays to determine the acceleration of wound healing which were carried out on selected clinical cases indicate that the formulations and methods of the present invention exhibit a significant beneficial result relative to traditional therapies. Specifically, in practice, it has been observed that chronically non-healing wounds, which were at least six months old prior to initial treatment, show new venules, arterioles and vascularity after only six days of treatment using the formulations and methods of the present invention. Years old, chronically non-healing wounds have been observed to close without scarring after about one to two weeks of treatment using the formulations and methods of the present invention.

No negative side effects have been observed through use of the formulations and methods of the invention. Particularly, in clinical trials performed to date on living human subjects, topical administration of the formulations of the present invention including insulin while effective for stimulating the healing of wounds did not influence the serum glucose levels in the patients treated therewith.

One positive side effect observed in the patients treated using the formulations and methods of the invention was a reduction in LDL cholesterol with a corresponding elevation in HDL cholesterol and a reduction in excess triglycerides.

The concepts of the invention will now be illustrated by the following Examples, which are intended to be purely exemplary and not limiting. The chemical debriding complex applied to the wound treated in example 1 was an aerosolized liquid composition comprising:

45 wt % sterile babasu oil,

29 wt % sterile avocado oil,

25 wt % (3%) hydrogen peroxide, and

1 wt % pramoxine hydrochloride (over the counter strength).

The chemical debriding complex applied to each of the wounds treated in examples 2 through 4 was an aerosolized liquid composition comprising:

30 wt % sterile babasu oil,

15 wt % sterile avocado oil,

25 wt % (3%) hydrogen peroxide,

1 wt % pramoxine hydrochloride (over the counter strength), and

29 wt % povidone iodine.

The formulation of the present invention used in the following examples was prepared in the form of a gel as follows:

(1) 1 ml of mixture (a) weighing about 100 g was prepared comprising:

200 Units of insulin,

500 mg hydrocortisone,

400 IU of tocopherol acetate,

800 IU of tocopherol lineolate,

1400 IU of retinyl palminate,

about 15 g ascorbyl glucosamine, and

a trace amount of Tenox-6 (anti-oxidant);

(2) 1 ml of mixture (b) weighing about 100 g was prepared comprising:

15 wt % sterile avocado oil,

35 wt % sterile babasu oil,

35 wt % sterile macadamia oil, and

15 wt % ethyl lineolate;

(3) 1 ml of mixture (c) weighing about 100 g was prepared comprising:

60.44 wt % sterile water,

10.00 wt % oat colloids,

6.00 wt % salicylic acid complex,

1.00 wt % wheat protein glyceride,

0.50 wt % zinc collagen,

2.00 wt % sea moss,

3.00 wt % glycerine,

0.20 wt % methyl paraban,

2.50 wt % SepiGel (emulsifier),

0.30 wt % ascorbyl glucosamine,

2.50 wt % cetyl ethanol,

1.50 wt % stearol ethanol,

3.00 wt % macadamia nut oil,

0.40 wt % silicone 5-5-6,

0.30 wt % tocopherol acetate,

0.50 wt % tocopherol lineolate,

0.30 wt % retinyl palmitate,

0.06 wt % Tenox-6 (anti-oxidant),

0.20 wt % Glydant Plus, and

0.30 wt % xanthum gum; and,

(4) 1 ml of mixture (a), 1 ml of mixture (b) and 1 ml of mixture (c) were combined using a three step homogenization process to form the gel formulation of the present invention used in the following examples, namely

(i) subjecting the combination of 1 ml of mixture (a), 1 ml of mixture (b) and 1 ml of mixture (c) to intense agitation continuously for about one hour;

(ii) allowing the combination to settle for about one hour, and

(iii) repeating (i) and (ii) twice.

The dose of formulation used in treating the wounds in the following examples was based on the wound volume. The wound volume was measured in cubic centimeters using well known methods.

### EXAMPLE 1

Old chronic ankle wound

A 97 year old, female, human patient in normal health, suffered from a 16 month old chronic interstitial, encapsulated, alpha-hemolytic, streptococcal channelling ulceration located on her right ankle and tunnelled to the periosteum of the bone in her right ankle. Conventional topical treatments, including Granulex®, zinc oxide, Duoderm® and Sorbasan® dressings, were used but were repeatedly unsuccessful. Conventional intravenous treatments, including a two week cycle of penicillin and a two week cycle of amoxicillin, were unsuccessful. Note that the penicillin and the amoxicillin were not administered during the same two week period. Conventional per orum treatments, including a two week cycle of Keflex® and a two week cycle of Cipro®, were unsuccessful. Note that the Keflex and Cipro were not administered during the same two week period, nor were they administered during the same two week period as either of the penicillin or the amoxicillin.

The wound was subsequently treated using the gel formulation prepared as described above. Specifically, the given formulation was administered to the patient's wound as part of the following treatment procedure. Particularly, (i) the wound was prepared for administration of the given formulation by liberal application of a chemical debriding complex described above; (ii) after allowing the chemical debriding complex to bubble off, about three (3) minutes, a medicated pad comprising a sterile pad containing about three (3) ml of the gel formulation for each cubic centimeter of wound volume was placed over the wound and secured to the patient; (iii) the medicated pad was removed about twenty-four (24) hours later; (iv) steps (i) through (iii) were repeated. After seven (7) days of treatment, the wound was completely closed.

### EXAMPLE 2

Old chronic back wound

A 95 year old, female, human patient in very poor health suffering from CHF, diabetes, peripheral vascular disease, 10% body surface non-candidyal yeast infection, nutritional deficit, and “steroid” skin secondary to prednisone administration for auto-immune arthritis and from an 18 month old chronic bed sore on the middle of her back. Conventional treatments, including granulex, zinc oxide, duoderm and sorbasan dressings in conjunction with two serial courses of oral antibiotics, per orum Cipro 500 mg B.I.D. followed by 500 mg intravenous Keflex every eight hours, were unsuccessful at stimulating the healing of the wound.

The wound was subsequently treated using a formulation and method of the invention. Specifically, the same formulation and method as described in relation to Example 1 above was used herein. After one treatment, the region of non-candidyal yeast infection treated completely disappeared. After six (6) day of treatment, the bed sore was completely closed.

### EXAMPLE 3

Canine pressure wound

A one year old, male Borzoi patient in a malnourished condition suffered from a six (6) month old chronic pressure-friction wound on both haunches directly under both sides of the anus. Conventional treatments, including granulex, zinc oxide, duoderm (post shaving) and sorbasan dressings, were used but were repeatedly unsuccessful.

The wound was subsequently treated using a formulation and method of the invention. Specifically, the same formulation and method as described in relation to Example 1 above was used herein. Seven (7) days after this treatment commenced, the wound was completely closed.

### EXAMPLE 4

Reptilian skin burn

A one (1) year old, Mexican iquana in dehydrated condition, suffered from a dime sized group of burns on all four of its paws and a quarter size burn on its stomach caused by overexposure to the sun.

The wounds were treated using a formulation and method of the invention. Specifically, the same formulation and method as described in relation to Example 1 above was used herein. After one (1) day of treatment, the wound was completely healed without any noticeable scaring.

The formulation used in the preceding examples may preferably be diluted for use on less sever wounds (see Table 1). Specifically, the above formulation may, for example, be diluted with at least one of oils and lipids, preferably oils.

TABLE 1

| | Conc. of diluting |
|---|---|
| Type or category of wound | liquid in formulation |
| 1. minor burns, including sunburn; | 99.95 vol. % to 97.5 vol. % |
| 2. burns which blister; and, | 97.5 vol. % to 92.5 vol. % |
| 3. detergent burns. | 92.5 vol. % to 90 vol. % |

While certain presently preferred embodiments of the invention have been illustrated and described, it is to be understood that the invention is not limited thereto and may be otherwise practiced within the scope of the following claims.

We claim:

1. A formulation for stimulating healing of a wound in a patient, consisting essentially of a pharmaceutically effective combination of insulin, hydrocortisone and a nutrient filled medium.

2. The formulation of claim 1, wherein the formulation contains a topical anesthetic.

3. The formulation of claim 1, wherein the formulation contains a preservative.

4. The formulation of claim 1, wherein the insulin is of a purity suitable for intravenous use in a human patient.

5. The formulation of claim 1, wherein the hydrocortisone is of a purity suitable for intravenous use in a human patient.

6. The formulation of claim 1, wherein the wound is a pressure wound.

7. The formulation of claim 6, wherein the formulation includes at least 200 Units of insulin per cubic centimeter of wound.

8. The formulation of claim 6, wherein the formulation includes at least 500 mg of hydrocortisone per cubic centimeter of wound.

9. The formulation of claim 1, wherein the formulation is a topical pharmaceutical composition selected from the group consisting of a lipid solution, a gel, a foam, a cream, a paste, a lotion, a spray, a suspension, a dispersion, a salve and an ointment.

10. A medicated pad, comprising a sterile pad containing a formulation consisting essentially of a pharmaceutically effective combination of insulin, hydrocortisone and a nutrient filled medium.

11. The medicated pad of claim 10, further comprising means for attaching the medicated pad to a patient.

12. A method for stimulating healing of a wound in a patient, comprising contacting the wound with a formulation consisting essentially of a pharmaceutically effective combination of insulin, hydrocortisone and a nutrient filled medium.

13. The method of claim 12, wherein the nutrient filled medium consists essentially of vitamins, proteins, lipids, carbohydrates and oils.

14. The method of claim 12, wherein the formulation further consists of a topical anesthetic.

15. The method of claim 12, wherein the formulation further consists of a preservative.

16. The method of claim 12, wherein the patient may be selected from the group consisting of humans, non-human mammals, and reptiles.

17. The method of claim 12, wherein the insulin is of a purity suitable for intravenous use in a human patient.

18. The method of claim 12, wherein the hydrocortisone is of a purity suitable for intravenous use in a human patient.

19. The method of claim 12, wherein the formulation is a topical pharmaceutical composition selected from the group consisting of a lipid solution, a gel, a foam, a cream, a paste, a lotion, a spray, a suspension, a dispersion, a salve and an ointment.

20. A method for stimulating healing of pressure wound in a patient, comprising topically administering to the pressure wound a formulation consisting essentially of a pharmaceutically effective combination of
insulin,
hydrocortisone,
a topical anesthetic,
a source of vitamin A,
a source of vitamin C,
a source of vitamin E,
a source of protein,
a source of carbohydrates,
a source of lipids,
at least one oil, and
a preservative.

21. The method of claim 20, wherein the formulation contains at least 200 Units of insulin per cubic centimeter of wound.

22. The method of claim 20, wherein the formulation contains at least 500 mg of hydrocortisone per cubic centimeter of wound.

23. The method of claim 20, wherein the topical anesthetic is pramoxine hydrochloride.

24. The method of claim 20, wherein the preservative is Glydant Plus®.

25. The method of claim 20, wherein the source of vitamin A is at least one of retinyl palminate and avocado oil.

26. The method of claim 20, wherein the source of vitamin C is ascorbyl glucosamine.

27. The method of claim 20, wherein the source of vitamin E is at least one of tocopherol acetate, tocopherol linoleate, and avocado oil.

28. The method of claim 20, wherein the formulation is a topical pharmaceutical composition selected from the group consisting of a lipid solution, a gel, a foam, a cream, a paste, a lotion, a spray, a suspension, a dispersion, a salve and an ointment.

29. A method for stimulating healing of a wound in a patient, comprising:
(a) preparing the wound for treatment;
(b) removing from a hermetically sealed container a medicated pad consisting essentially of a sterile pad impregnated with a formulation consisting essentially of a pharmaceutically effective combination of insulin, hydrocortisone and a nutrient filled medium; and,
(c) covering the wound with the medicated pad.
